(19) United States
(12) Patent Application Publication
(10) Pub. No.: US 2023/0165392 A1
(43) Pub. Date: Jun. 1, 2023

(54) BEDDING COMPONENTS INCLUDING A DESICCANT

(71) Applicant: Dreamwell, Ltd., Doraville, GA (US)

(72) Inventors: Brian Mark Anderson, Atlanta, GA (US); Felipe Salvador Arias, Dunwoody, GA (US); Sheri L. McGuire, Duluth, GA (US)

(21) Appl. No.: 18/055,001

(22) Filed: Nov. 14, 2022

Related U.S. Application Data
(60) Provisional application No. 63/284,224, filed on Nov. 30, 2021.

(51) Int. Cl. A47G 9/00; A47C 31/00; A47C 27/14; A47G 9/10

(52) U.S. Cl. CPC A47G 9/007; A47C 31/007; A47C 27/142; A47G 9/10

(57) ABSTRACT

Bedding components generally include a desiccant applied or infused into a selected bedding component. The desiccant can be microencapsulated, applied as a topical solution, and/or provided within a container including a moisture permeable membrane.

### CROSS-REFERENCE TO RELATED APPLICATIONS

This application claims the benefit of U.S. Provisional Application No. 63/284,224, filed Nov. 30, 2021, the disclosure of which is incorporated herein by reference in its entirety.

### BACKGROUND

The present disclosure generally relates to bedding components including a desiccant configured to reduce moisture and modulate moisture content within a given microclimate in which the bedding components are situated.

Thermal discomfort during sleep is a common problem that affects various bedding components including, for example, mattress, sheeting, pillows, and the like. There are two primary factors that contribute to this discomfort, which generally include heat buildup and moisture buildup such as may occur from exposure to humidity and/or from the end users themselves. Several solutions (passive and active) are known to address the heat aspect of comfort including integration of thermally conductive fabrics and foams into the bedding component, integration of phase change materials onto or proximate to contact surfaces, as the use of blowers to force ambient air into the microclimate (area between the covers and mattress where the person sleeps). However, with regard to moisture buildup, the solutions are somewhat limited and generally include active solutions such as, for example, forcing ambient air into the microclimate. There are no known effective passive methods for reducing moisture content such as may occur in a high humidity microclimate prior to condensation.

### BRIEF SUMMARY

Disclosed herein are bedding components, mattress assemblies and pillows generally including a desiccant. In one embodiment, the bedding component includes a mattress assembly, a sheet overlying the mattress assembly, and one or more pillows provided on the sheet corresponding to a head position of the mattress assembly; and a desiccant on and/or within one or more selected surfaces of the mattress assembly, the sheet, and/or the one or more pillows.

In one embodiment, a mattress assembly includes at least one layer including a surface; and a desiccant on at least portions of the surface and/or in the at least one layer.

In another embodiment, a pillow includes an outer envelope; a cushioning material within the envelope wherein the cushioning material comprises a plurality of feathers, foam, and/or a plurality of fibers within the envelope; and a desiccant on at least portions of the surface and/or in the at least one layer.

The disclosure may be understood more readily by reference to the following detailed description of the various features of the disclosure and the examples included therein.

### BRIEF DESCRIPTION OF THE DRAWINGS

Example embodiments of the invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

Figure (FIG. 1 illustrates a cross sectional view of an exemplary mattress assembly including a desiccant material in accordance with an embodiment of the present disclosure.

FIG. 2 illustrates a cross sectional view of an exemplary mattress assembly including a desiccant material in accordance with an embodiment of the present disclosure in accordance with an embodiment of the present disclosure.

FIG. 3 illustrates a cross sectional view of an exemplary mattress assembly including a desiccant material in accordance with an embodiment of the present disclosure in accordance with an embodiment of the present disclosure.

FIG. 4 illustrates a cross sectional view of an exemplary mattress assembly including a desiccant material in accordance with an embodiment of the present disclosure in accordance with an embodiment of the present disclosure.

FIG. 5 illustrates a cross sectional view of an exemplary pillow including a desiccant material in accordance with an embodiment of the present disclosure in accordance with an embodiment of the present disclosure.

### DETAILED DESCRIPTION

Disclosed herein are bedding components such as sheeting, pillows, mattresses, and the like that include a desiccant integrated therein and/or thereon configured to reduce and/or modulate moisture content within a given microclimate in which the particular bedding component is situated. The desiccant can be applied and/or inserted with any of the layers defining the bedding component. As will be described in greater detail below, the desiccant is generally a hydroscopic substance that induces or sustains a state of dryness (desiccation) in its local vicinity. Some desiccants may be solids that generally function by absorption or adsorption of water, or a combination of water absorption and adsorption. Exemplary desiccants may include salts, molecular sieves, activated charcoal, and the like. The particular desiccant or mixture of desiccants is not intended to be limited. By way of example, suitable desiccants can include silica gel; activated alumina; molecular sieves (crystalline aluminosilicates); montmorillonite clay; salts such as sodium phosphate di-basic, potassium carbonate, magnesium chloride, calcium oxide, calcium sulfate, and the like; activated charcoal; combinations thereof; and/or the like.

In one or more embodiments, the desiccant can be provided a solution and topically applied to a selected surface. In other embodiments, the desiccant can encapsulated. For example, individual grains or a liquid of the desiccant can be microencapsulated within a moisture permeable membrane and applied to a selected surface of the bedding component, in still other embodiments, a larger amount the desiccant relative to microencapsulation can be contained within a removable and replaceable moisture permeable membrane. The moisture permeable membrane can be flexible or inflexible depending on the application. The desiccant can b in the form of granules and/or can be microencapsulated. Advantageously, implementing a desiccant on and/or within the bedding component enables a passive solution to reduce moisture within the microclimate about the bedding component. Moreover, reducing moisture can prevent mold formation and odors.

As noted above, the particular desiccant is not intended to be limited and can include various desiccants that can be applied to and/or infused in the various mattress components (fabric, fiber, foam, etc.) or accessories through chemical bonding, spraying of microencapsulated desiccant, insertion of moisture permeable containers of the desiccant or desiccants, or the like. In one or more embodiments, the desiccant is selected to have a saturation point between typical room humidity and the microclimate humidity to ensure maximum effectiveness. This enables the desiccant to absorb moisture when a person sleeps in proximity to the desiccant as well as release moisture to the environment in the absence of a person.

For the purposes of the description hereinafter, the terms “upper”, “lower”, “top”, “bottom”, “left,” and “right,” and derivatives thereof shall relate to the described structures, as they are oriented in the drawing figures. The same numbers in the various figures can refer to the same structural component or part thereof. Additionally, the articles “a” and “an” preceding an element or component are intended to be nonrestrictive regarding the number of instances (i.e. occurrences) of the element or component. Therefore, “a” or “an” should be read to include one or at least one, and the singular word form of the element or component also includes the plural unless the number is obviously meant to be singular.

Spatially relative terms, e.g., “beneath,” “below,” “lower,” “above,” “upper,” and the like, can be used herein for ease of description to describe one element or feature's relationship to another element(s) or feature(s) as illustrated in the figures.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

As used herein, the term “about” modifying the quantity of an ingredient, component, or reactant of the invention employed refers to variation in the numerical quantity that can occur, for example, through typical measuring and liquid handling procedures used for making concentrates or solutions. Furthermore, variation can occur from inadvertent error in measuring procedures, differences in the manufacture, source, or purity of the ingredients employed to make the compositions or carry out the methods, and the like.

It will also be understood that when an element, such as a layer, region, or substrate is referred to as being “on” or “over” another element, it can be directly on the other element or intervening elements can also be present. In contrast, when an element is referred to as being “directly on” or “directly over” another element, there are no intervening elements present, and the element is in contact with another element.

Turning now to FIGS. 1-3, there are shown various cross-sectional views of exemplary mattress assemblies 10, 50 and 100 including one or more desiccants provided on and/or infused into a selected one or more surfaces of the mattress assembly. As shown in FIGS. 1-3, the mattresses 10, 50 and 100 generally have in common a base layer 12, an inner core layer 14 on the base layer 12, and at least one layer 16 overlying the inner core layer 14. A side rail assembly (not shown) may be disposed about at least a portion of the perimeter of the different layers 12, 14, and 16. The mattresses can be covered with any suitable covering or sheet material or materials, padding or contour materials, quilting, or topper layers, and nay also incorporate fire resistant materials. The exemplary mattress assemblies 10, 50, 100 are not intended to be limited to the particular mattress assemblies as depicted and can include more or less layers depending on the intended application. Any of the described mattresses herein can be made in any desired shape or size, including for example conventional bed sizes such as single, twin, double; queen and king.

The one or more layers, e.g., layers 12, 14, 16 as shown can generally be a coil spring layer, e.g., pocketed coil springs, synthetic or natural foam layers, synthetic or natural fiber layers, or combinations thereof. In the various mattress assemblies 10, 50, 100, a desiccant 18 is provided in and/or on selected surfaces thereof.

The coil springs may be open coils or may be encased coils, e.g., pocketed (Marshall) coils. In some embodiments, the coil spring layer may further include foam.

The foam utilized in the mattress assemblies is not intended to be limited and can include polyurethane foams, latex foams including natural, blended and synthetic latex foams; polystyrene foams, polyethylene foams, polypropylene foam, polyether-polyurethane foams, and the like. Likewise, the foam layer can be selected to be formed from viscoelastic or non-viscoelastic foams. Some viscoelastic materials are also temperature sensitive, thereby also enabling the foam layer to Change hardness/firmness based in part upon the temperature of the supported part. Unless otherwise noted, any of these foams may b′ open cell or closed cell or a hybrid structure of open cell and closed cell. Likewise, the foams can be reticulated, partially reticulated or non-reticulated foams. The term reticulation generally refers to removal of cell membranes to create an open cell structure that is open to air and moisture flow. Still further, the foams may be gel infused in some embodiments. In some embodiments, the foam layer can include One, or more channels (not shown) for improved air flow. In the one or more embodiments including more than one foam layer, each of the multiple foam layers can be formed of the same, material configured with different properties or can be formed of different materials.

The various foams suitable for use in the foam layer may be produced according to methods known to persons ordinarily skilled in the art. For example, polyurethane foams are typically prepared by reacting a polyol with a polyisocyanate in the presence of a catalyst, a blowing agent, one or more foam stabilizers or surfactants and other foaming aids. The gas generated during polymerization causes foaming of the reaction mixture to form a cellular or foam structure. Latex foams are typically manufactured by the well-known Dunlap or Talalay processes. Manufacturing of the different foams are well within the skill of those in the art.

The different properties for each foam layer may include, but are not limited to, density, hardness, thickness, support factor, flex fatigue, air flow, various combinations thereof, and the like. Density is a measurement of the mass per unit volume and is commonly expressed in pounds per cubic foot. Density can be and is measured in accordance with ASTM D-3574. The hardness properties of foam are also referred to as the indention load deflection (ILD) or indention force deflection GM) and is also measured in accordance with ASTM D-3574. In the case of multiple foam layers, the density and hardness properties can be varied or the same. Moreover, combinations of properties may be varied for each individual layer. The individual foam layers can also be of the same thickness or may have different thicknesses as may be desired to provide different tactile responses.

In one or more embodiments, the hardness property of the foam layers generally have an ILD within a range of about 35 to about 100 pounds-force. In other embodiments, the hardness property of the foam layers can have an ILD of about 40 to about 90 pounds-force; and in still other embodiments, the hardness property of the foam layers can have an ILD of about 50 to about 75 pounds-force. In one or more embodiments, the density of the foam layers can generally range from about 1 to about 3 pounds per cubic foot (pcf). In other embodiments, the density of the foam layers can range from about 1.2 to about 2 pcf.

The side rail assembly may be attached to or placed adjacent to at least a portion of the perimeter of the mattress assemblies, and may include metal springs, spring coils, encased spring coils, foam, latex, natural latex, latex w/ gel, gel, viscoelastic gel, or a combination, in one or more layers. For example, side rails may be placed on one or more of the sides of the mattress assembly, e.g., on all four sides of the stacked mattress layers, on opposing sides, on three adjacent sides, or only on one side of the stacked mattress layers. In certain embodiments, the side rails may include edge supports with a firmness greater than that provided by the mattress assembly layers 12, 14, 16. The side rails may be fastened to the mattress layers via adhesives, thermal bonding, or mechanical fasteners.

As shown in FIG. 1, the desiccant 18 can be applied as a coating and/or as a self-contained flexible package and/or as microencapsulates on and/or in selected portions of one or more surfaces of the mattress assembly. For example, as shown, the desiccant 18 is provided on inner core layer 14 and can be spaced apart at about equal increments. In other embodiments, the desiccant 18 can be provided in different zones of the mattress assembly such as those zones subject to the greatest amounts of moisture when in use. In still other embodiments, the desiccant can be provided on multiple surfaces at different vertical locations relative to the sleeping surface. It should be apparent that the particular location is not intended to be limited.

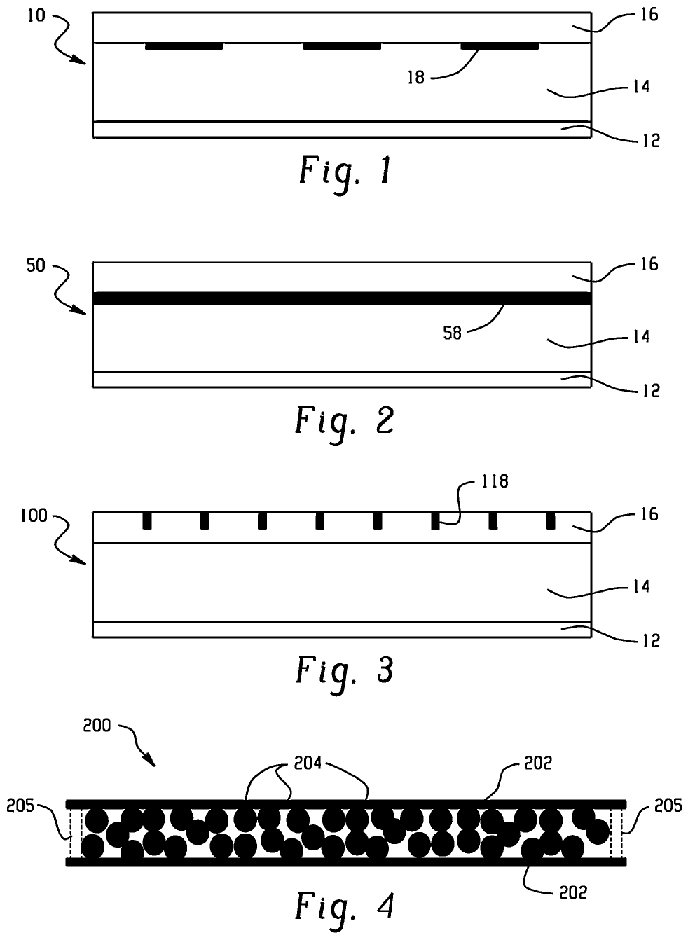

Referring now to FIG. 2, the mattress assembly 50 includes application of the desiccant onto an entire surface of one or more of the layers defining the mattress assembly, e. g., on a bottom surface of layer 16 or on a top surface of layer 14. In some embodiments, the desiccant 58 can be applied onto the entire surface albeit in combination with another desiccant on a different layer such as described in FIG. 1. The desiccant can be the same or different. Likewise, a mixture of different desiccants can be used. Still further, the desiccant can be infused into the layer or can be applied on as well as infused into the selected layer.

In FIG. 3, mattress assembly 100 include application of a desiccant into slot openings within a selected layer. The desiccant can be topically applied into the slot and/or can be contained within a moisture permeable container. In this manner, the desiccant 118 can wick moisture from a selected surface into the body of the layer, uppermost layer 16.

As noted above, the desiccants 18, 58, 118 can be topically applied as a solution and/or infused into the selected surface or prepackaged as an insert into the respective mattress assembly. By way of example, the desiccant can be in the form a solvent based solution, sprayed onto a selected surface and subsequently dried by heating above the boiling point of the solvent. The solvent is not intended to be limited and is generally non-reactive to the material defining the applied surface and has a boiling point sufficient for efficient and effective removal.

Alternatively, the desiccant can be prepackaged such as is shown in FIG. 4. The package 200 generally includes a moisture permeable membranes 202 attached at ends 205 to encapsulate the desiccant 204 so as to permit absorption or adsorption of water molecules onto the desiccant, which may or may not be flexible depending on the intended application. For example, the desiccant can be a silica gel pack including a plurality of grains 204 of the desiccant such as silicon dioxide disposed within a moisture permeable membrane. In some embodiments, the moisture permeable membrane includes a plurality of perforations, wherein the grains of desiccant have a diameter greater than the perforations.

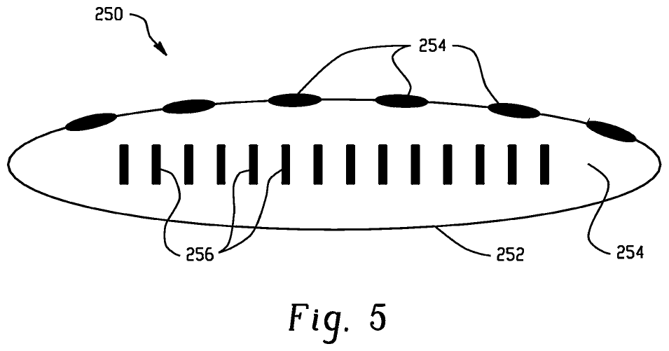

Turning now to FIG. 5, there is depicted a cross sectional view of a bedding component in the form of a pillow 250. The pillow 250 generally includes an outer envelope 252; and a cushioning material 254 encapsulated within the envelope 252, wherein the outer envelope can optionally include a topically applied and/or infused desiccant 254 and/or a packaged desiccant 256 within the cushioning material 254. The cushioning material 254 is not intended to be limited and can include foam, feathers, fiber padding or the like. Optionally, the envelope 252 can include a zippered opening to access the cushioning material 254. The pillow 250 can further include a pillowcase as may be desired for aesthetic and comfort purposes. The pillowcase as well as any sheets or other bedding components used in the mattress assembly or during use of the mattress can also be treated with the desiccant in a similar manner to that described above.

Advantageously, implementing the desiccant in the mattress assembly or accessories enables a passive solution to solve the moisture problem, which will generally be cheaper and more effective than active solutions. The mattress assembly with the desiccant as described herein will provide enhanced comfort by reducing the sleeper's perceived temperature.

It should be apparent that the desiccant as described above can be integrated into various linens used in conjunction with the mattress assemblies. Likewise, the various layers defining a mattress assembly as generally described above can include the desiccant including but not limited to the fabric layers, the fire-retardant sock, border ticking, backing layers, and the like.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A mattress assembly, comprising:
at least one layer including a surface; and
a desiccant on at least portions of the surface and/or in the at least one layer.

2. The mattress assembly of claim 1, wherein the at least one layer comprises a foam layer, a coil layer, a fiber layer or combinations thereof.

3. The mattress assembly of claim 1, wherein the desiccant is selected to have a saturation point greater than an ambient relative humidity about a microclimate of the mattress assembly.

4. The mattress assembly of claim 1, wherein the desiccant is applied to different zones of the mattress.

5. The mattress assembly of claim 1, wherein the desiccant comprises a moisture permeable container and a plurality of desiccant grains within the moisture permeable container.

6. The mattress assembly of claim 1, wherein the desiccant is microencapsulated within a moisture permeable membrane.

7. The mattress assembly of claim 1, wherein the at least one layer comprises one or more slot openings, and wherein the desiccant is packaged into a container comprising a moisture permeable membrane and is provided within the one or more slot openings.

8. A pillow comprising
an outer envelope;
a cushioning material within the envelope wherein the cushioning material comprises a plurality of feathers, foam, and/or a plurality of fibers within the envelope; and
a desiccant on at least portions of the surface and/or in the at least one layer.

9. The pillow of claim 8, wherein the desiccant is packaged into a container comprising a moisture permeable membrane and is provided within the one or more slot openings within the cushioning material.

10. The pillow of claim 8, wherein the desiccant is selected to have a saturation point greater than an ambient relative humidity about a microclimate of the mattress assembly.

11. The pillow of claim 8, wherein the desiccant is on a surface of the cushioning material and/or within the cushioning material.

12. The pillow of claim 8, wherein the desiccant comprises a moisture permeable container and a plurality of desiccant grains within the moisture permeable container.

13. The pillow of claim 8, wherein the desiccant is microencapsulated within a moisture permeable membrane.

14. The pillow of claim 8, wherein the cushioning material comprises one or more slot openings, and wherein the desiccant is packaged into a flexible container comprising a moisture permeable membrane and is provided within the one or more slot openings.

15. A bedding component, comprising:
a mattress assembly, a sheet overlying the mattress assembly, and one or more pillows provided on the sheet corresponding to a head position of the mattress assembly; and
a desiccant on and/or within one or more selected surfaces of the mattress assembly, the sheet, and/or the one or more pillows.

16. The bedding component of claim 15, wherein the desiccant is packaged into a container comprising a moisture permeable membrane and is provided within the one or more slot openings within the cushioning material.

17. The bedding component of claim 15, wherein the desiccant is selected to have a saturation point greater than an ambient relative humidity about a microclimate of the mattress assembly.

18. The bedding component of claim 15, wherein the desiccant is on a surface of the cushioning material and/or within the cushioning material.

19. The bedding component of claim 15, wherein the desiccant comprises a moisture permeable container and a plurality of desiccant grains within the moisture permeable container.

20. The bedding component of claim 15, wherein the desiccant is microencapsulated within a moisture permeable membrane.

21. The bedding component of claim 15, wherein the cushioning material comprises one or more slot openings, and wherein the desiccant is packaged into a flexible container comprising a moisture permeable membrane and is provided within the one or more slot openings.
